(19) United States
(12) Patent Application Publication
(10) Pub. No.: US 2007/0112293 A1
(43) Pub. Date: May 17, 2007

(54) Method for shunting toxic substances from a brain ventricle to the sinus system

(75) Inventor: Svend Erik Borgesen, Kokkedal (DK)

Correspondence Address: BROWDY AND NEIMARK, P.L.L.C.;624 NINTH STREET, NW, SUITE 300, WASHINGTON, DC, 20001-5303, US

(73) Assignee: SINU SHUNT A/S, Glostrup (DK)

(21) Appl. No.: 10/580,649

(22) PCT Filed: Nov. 26, 2004
(86) PCT No.: PCT/DK04/00827

§ 371 Date: May 25, 2006

Related U.S. Application Data
(60) Provisional application No. 60/524,887, filed on Nov. 26, 2003.

(30) Foreign Application Priority Data
Nov. 26, 2003 (DK) PA 2003 01749

(51) Int. Cl. A61M 5/00

(52) U.S. Cl. 604/9

(57) ABSTRACT

The present invention is directed to methods for controlled and optimized removal of cerebrospinal fluid (CSF) from the CSF space of a patient. The methods are particularly intended for the treatment of Alzheimer's disease and other conditions which are caused by, or otherwise related to, the retention and/or accumulation of toxic substances in the CSF. One aspect of the present invention provides a method for shunting toxic substances present in a brain ventricle to the sinus system of an individual suffering from, or at risk of developing, a condition related to the retention and/or accumulation of toxic substances in the CSF, such as Alzheimer's disease. In addition to Alzheimer's disease, the present invention will be useful for treating other conditions resulting from the accumulation of toxic substances and resulting lesions in the patient's brain, such as Down's Syndrome, hereditary cerebral hemorrhage with amyloidosis of the Dutch-Type (HCHWA-D), epilepsy, narcolepsy, Parkinson's disease, polyneuropathies, multiple sclerosis, amyotrophic lateral sclerosis (ALS), myasthenia gravis, muscular dystrophy, dystrophy myotonic, other myotonic syndromes, polymyositis, dermatomyositis, brain tumors, Guillain-Barre-Syndrome, and the like.

All patent and non-patent references cited in the present patent application is hereby incorporated in their entirety. This application is a non-provisional of U.S. provisional application Ser. No. 60/524,887 filed 26 Nov. 2003, which is hereby incorporated by reference in its entirety.

### FIELD OF INVENTION

The present invention relates to a method for shunting toxic substances, present in brain tissue and/or the CSF space, such as present in a brain ventricle, to the sinus system of an individual suffering from, or at risk of developing, a condition related to the retention and/or accumulation of toxic substances in the brain tissue and/or the CSF space.

### BACKGROUND OF INVENTION

Cerebrospinal Fluid

The brain and spinal cord are bathed in cerebrospinal fluid (CSF) and encased within the cranium and vertebral column inside a thin membrane known as the meninges. The space within the meninges includes the subarachnoid space, the ventricles (including the lateral ventricle, third ventricle, and fourth ventricle), the vertebral column, and the brain interstitial spaces. The volume of the brain intracranial spaces is on average about 1700 ml. The volume of the brain is approximately 1400 ml, and the volume of the intracranial blood is approximately 150 ml. The remaining 150 ml is filled with CSF (this volume may vary within 60 ml to 290 ml). The CSF circulates within the CSF space. Cerebrospinal fluid is formed in the ventricular system irrespective of the intracranial pressure (ICP). The formation rate is constant, with a range of 0.3-0.4 ml/min. (Børgesen and Gjerris 1987).

Under normal conditions, the CSF is produced in the chorioid plexus in the ventricles. It flows through the ventricles, aqueduct and basal cisterns over the cerebral surface to the arachnoid villi, from where the CSF is absorbed into the sagittal sinus (including sinus transversus). The production and absorption of CSF are well described in the medical literature. See, e.g., Adams et al. (1989) “Principles of Neurology,” pp. 501-502.

Articles discussing pressures and other characteristics of CSF in the CSF space include Condon (1986) J. Comput. Assit. Tomogr. 10:784-792; Condon (1987) J. Comput. Assit. Tomogr. 11:203-207; Chapman (1990) Neurosurgery 26:181-189; Magneas (1976) J. Neurosurgery 44:698-705; Langfitt (1975) Neurosurgery 22:302-320.

Alzheimer's Disease and Other Diseases Caused by Toxic Substances in the CSF

Alzheimer's disease (AD) is a degenerative brain disorder characterised clinically by progressive loss of memory, cognition, reasoning, judgement, and emotional stability and which gradually leads to profound mental deterioration and ultimately death. Alzheimer disease is the most common cause of progressive mental failure (dementia) in aged humans and is estimated to represent the fourth most common medical cause of death in the United States. Alzheimer's disease has been observed in all races and ethnic groups world wide and presents a major current and future public health problem. The disease is currently estimated to affect about two to four million individuals in the United States

A hallmark of AD is the accumulation in the brain of extracellular insoluble deposits called amyloid plaques, and abnormal lesions within neuronal cells called neurofibrillary tangles. The presence of amyloid plaques, together with neurofibrillary tangles, are the basis for definitive pathological diagnosis of AD. Increased plaque formation is associated with increased risk of AD.

A variety of other human diseases also demonstrate amyloid deposition. In Alzheimer's disease and other amyloid diseases, there is currently no cure or effective treatment, and the patient usually dies within 3 to 10 years from disease onset. Stimulated memory exercises on a regular basis have been shown to slow, but not stop, memory loss. A few drugs, such as tacrine, result in a modest temporary improvement of cognition but do not stop the progression of dementia.

Research on the molecular pathogenesis of Alzheimer's disease (AD) has resulted in a protein chemical analysis of two extracellular and intracellular fibrillary lesions in AD brain, containing beta-amyloid protein (Abeta) and tau as their major components, respectively.

Linkage analysis of familial AD identified four responsible genes: three causative genes (beta-amyloid precursor protein (APP), presenilin 1, and presenilin 2) and one susceptibility gene (apolipoprotein E epsilon4). All those genes causing and predisposing to AD exhibit a common phenotype: an increased production of Abeta42, a longer, more amyloidogenic Abeta species, and/or its enhanced deposition. This observation was substantiated when presenilins were shown to be directly involved in Abeta production.

Whereas Abeta deposition is relatively specific for AD, tau deposition is observed in various neurodegenerative diseases and is assumed to be intimately associated with neuronal loss. Genetic analysis of frontotemporal dementia and parkinsonism linked to chromosome 17 (FTDP-17) revealed the presence of mutations in the tau gene in affected members. Thus, tau can lead to intracellular tau deposits and neuronal loss.

Taken together, Abeta might exert neurotoxicity through tau, leading to neuronal loss in the AD brain. (Morishima-Kawashima M, Hara Y,“Alzheimer's disease: beta-amyloid protein and tau”, Journal of Neuroscience research” 70 (3): 392-401, Nov. 1 2002; “Amyloid precursor protein (APP) and the biology of proteolytic processing: relevance to Alzheimer's disease”. Int J Biochem Cell Biol, 2003;35(11):1505-1535).

Beta-2 microglobulin is another example of a protein whose concentration in the cerebrospinal fluid increases with age and reaches high levels in patients with adult-onset dementia of the Alzheimer's type. (Martinez et al., (1993) “Relationship of interleukin-1 beta and beta-2-microglobulin with neuropeptides in cerebrospinal fluid of patients with dementia of the Alzheimer type,” J. Neuroimmunology 48: 235-240). Beta-2 microglobulin is associated with amyloid deposits in some tissues of patients on long-term renal hemodialysis. (Ono et al., (1994) “Formation of amyloid-like substance from beta-2-microglobulin in vitro. Role of serum amyloid P component: a preliminary study,” Nephron 66: 404-407).

It is has been suggested (Rubenstein (1998) The Lancet, 351:283-285) that Alzheimer'disease may be treated by removal of cerebrospinal fluid (CSF) from the CSF space of a patient suffering from Alzheimer's disease. This proposal is based on the suggestion that in at least some cases, the characteristic lesions, referred to as amyloid plaques, and other characteristic lesions in the brain associated with Alzheimer's disease, result from the retention of certain toxic substances in the CSF space of the patient. Rubenstein states that “this hypothesis can be tested by assessing the long-term effects of an implanted flow-controlled ventriculoperitoneal shunt (VP shunt) on the concentration of various CSF solutes”.

Prior art methods for shunting toxic proteins from the CSF space relate exclusively to ventriculoperitoneal shunting methods and do not disclose shunting to the sagittal sinus or transverse sinus, nor do they disclose the use of a constant, essentially passive resistance to CSF flow to control the flow of CSF from the ventricles.

U.S. Pat. No. 5,980,480 describes a method for treating a patient for adult-onset dementia of the Alzheimer's type by removing a portion of the patient's cerebrospinal fluid by transporting the fluid to another portion of the patient's body. However, the method disclosed in U.S. Pat. No. 5,980,480 does not employ a step of shunting CSF to the sinus system, including the saggital sinus. U.S. Pat. No. 5,980,480 is exclusively directed to a method for shunting to e.g. the peritoneal cavity. However, shunting to areas other than the sagittal sinus or transverse sinus leads to the problem of posture related changes in the differential pressure across the shunt. Thus, the method described in U.S. Pat. No. 5,980,480 requires pressure regulation within the shunt system to compensate for alterations in pressure differences between the ventricles and resorption site. The methods of the present invention does not employ any pressure regulation means.

The treatment of Alzheimer's disease by shunting cerebrospinal fluid from the CSF region of the brain is also described in U.S. Pat. No. 6,575,928 and U.S. Pat. No. 6,383,159. Again, in these inventions the CSF removal rate is achieved by “providing a pressure-controlled variable resistance path in the flow control module between the CSF space and the disposal site”. The sagittal sinus or the transverse sinus are not described as resorption sites. The present invention does not rely on control of flow via “pressure responsive valves”, but functions on an entirely different principle: Maintenance of a passive and essentially constant resistance to the flow of CSF comprising toxic substances.

The present invention is not concerned with VP shunting methods as will be clear from the below disclosure of the invention.

### SUMMARY OF THE INVENTION

There is a need for an improved method for removing CSF from the CSF space of a patient in a treatment for Alzheimer's disease and other conditions relating to the presence and/or build up of toxic substances in cerebrospinal fluids.

Alzheimer's disease is not associated with increased intracranial pressure as is often observed in hydrocephalus. In contrast, the ICP is normal or even low. So is the resistance to outflow when measured by standard techniques. The reason why the toxic substances are accumulated in e.g. Alzheimer's disease is not fully understood. A conceivable explanation seems to be that while the CSF and its normally contained substances are resorbed at normal rates (i.e. with a normal resistances to outflow) the larger molecules of the toxic substances are not able to pass the normal resorption routes.

Therefore, if CSF is to be drained via another system than the normal resorption routes (i.e. arachnoid villi or maybe transcapillary) the imposed (implanted) drainage device must have a lower than normal resistance to outflow and also an opening pressure not exceeding the pressure in the normal receiving compartment for the CSF, i.e. the cranial venous sinus systems.

Conventional VP shunts are designed to drain CSF when there is an excess of CSF accumulated due to defects in the normal resorption mechanisms. By including flow restriction means, such as for example opening-pressure devices or pressure regulated variable resistance controls, drainage of CSF takes place until the desired pressure level is obtained.

In Alzheimer's disease the normal CSF resorption is still functioning and implanting a conventional VP shunting device will not lead to drainage of possible toxic substances. There is no excess CSF-accumulation and no increases in ICP.

The methods of the present invention exploit a drainage device for draining toxic substances in e.g. Alzheimer's disease offer a CSF-outflow at a lower outflow resistance than the normal level. If this is done by draining to a site with pressure levels lower than the intended intracranial pressure, such as the peritoneum or the atrium, the result will be over-drainage of CSF leading to the severe symptoms and complications related to over-drainage.

By shunting to the cranial sinuses the ICP is automatically ensured not to reach a level lower than the pressure in the sinus. Over drainage is thereby avoided. By shunting to the sinuses it is thus possible to offer CSF outflow via an outflow route with a low resistance to outflow of CSF, thereby making it possible to drain CSF containing toxic substances.

In summary, whereas conventional ventriculoperitoneal (VP) shunts are designed for use in treating normal pressure hydrocephalus, a method for treating normal pressure hydrocephalus cannot be used indiscriminately for draining toxic substances from the CSF space as intracranial pressures and specific flow control characteristics are very different in methods for treating normal pressure hydrocephalus and methods for shunting toxic CSF proteins in patients with e.g. Alzheimer's disease.

The method of the present invention will preferably provide for the controlled removal of CSF from the CSF space in a manner which effectively ensure that the amount of toxic substances is reduced without excessive removal of the CSF. The present invention solves this problem. The solution is provided by shunting toxic substances in the CSF to the sinus system by using a passive and essentially constant resistance to outflow. This is possible only by shunting the toxic substances to the sinus system as the differential pressure between the ventricles and the sinus is essentially constant. Accordingly, there is no need for pressure sensitive valves operating with an opening pressure as disclosed for prior art VP shunts. The elimination of pressure sensitive valves operating with a predefined opening pressure also makes it possible to drain toxic substances at a lower intracranial pressure than that at which the prior art shunt valves would not have able to ensure CSF drainage. Accordingly, the prior art VP shunts cannot be used in the methods of the present invention.

In the present invention, the CSF flow rate is controlled merely by maintaining a constant resistance to flow. This is enabled by preferably using the saggital sinus or the transverse sinus as a resorption site, which allows the pressure difference over the CSF shunt system to remain essentially constant. This would not be the case for resorption sites such as the peritoneum. Furthermore, the pressure difference generated across the shunt is similar to the low physiological pressure differences between the ventricles and the normal CSF resorption site in patients suffering e.g. from Alzheimer's disease.

Another problem caused by current VP shunting methods for shunting toxic substances from the CSF is that as the toxic substances flow through the shunt, they tend to adhere to the shunt sides. One solution to this problem could be to adapt currently used shunts, suitable for shunting toxic proteins, to have a larger internal diameter. Another alternative would be to adapt currently used shunts for shunting toxic proteins to have a shorter distance between the CSF and resorption site. There are however problems with these ideas, as currently used shunts for shunting toxic proteins use resorption sites far from the brain, such as the peritoneum. Current solutions to the problem of adhesion of the toxic proteins to shunts are pressure regulation systems, including pumps, which aid in forcing the flow of the toxic proteins through the shunt.

Methods according to the present invention provide for the controlled and optimized removal of cerebrospinal fluid (CSF) from the CSF space of a patient. The methods are particularly intended for the treatment of Alzheimer's disease and other conditions which are caused by, or otherwise related to, the retention and/or accumulation of toxic substances in the CSF.

In addition to Alzheimer's disease, the present invention will be useful for treating other conditions resulting from the accumulation of toxic substances and resulting lesions in the patient's brain, such as Down's Syndrome, hereditary cerebral hemorrhage with amyloidosis of the Dutch-Type (HCHWA-D), epilepsy, narcolepsy, Parkinson's disease, polyneuropathies, multiple sclerosis, amyotrophic lateral sclerosis (ALS), myasthenia gravis, muscular dystrophy, dystrophy myotonic, other myotonic syndromes, polymyositis, dermatomyositis, brain tumors, Guillain-Barre-Syndrome, and the like.

Down's Syndrome

In one preferred embodiment of the present invention, a method for treatment of Down's syndrome is provided. Nearly all patients with Down's syndrome develop Alzheimer's if they live into their 40s. This is probably due to the finding that APP is located on chromosome 21, a key chromosome in the genetic aberrations causing Down's syndrome patients. Thus, it is probable that Down's syndrome patients with genetic aberrations such as trisomy 21 will overproduce APP and have high levels of potentially toxic amyloid precursors in their CSF.

Hereditary Cerebral Hemorrhage with Amyloidosis of the Dutch-Type (HCHWA-D)

Hereditary cerebral haemorrhage with amyloidosis-Dutch type (HCHWA-D) is an autosomal dominant disorder, caused by a single base mutation in the APP gene, resulting in recurrent haemorrhagic strokes and dementia (Brain. 1997 December;120 (Pt 12):2243-9. It is envisaged that HCHWA-D and similar diseases caused by mutations in the APP gene may be treated using the methods described herein.

Epilepsy

In another, equally preferred embodiment of the present invention, a method for treatment of epilepsy is provided. Epilepsy is the tendency to have repeated seizures that originate in the brain. There are various toxic factors that may act to increase the risk of seizure. Increased levels of messenger RNAs for neurotrophic factors have been detected in brains during kindling epileptogenesis (Ernfors P, et al., Neuron. 1991 July;7(1):165-76) and this is hypothesised to contribute to the development of epileptic syndromes. Furthermore, increases in the levels of the excitory neurotransmitter glutamate, which in turn triggers increases in calcium ions to toxic levels, may also contribute to seizure occurrence.

Parkinson's disease

In another, equally preferred embodiment of the present invention, a method for treatment of Parkinson's disease is provided. Two different alpha-synuclein mutations have been shown to be associated with autosomal-dominant Parkinson's disease (PD), and the discovery that alpha-synuclein is a major component of Lewy bodies and Lewy neurites, the pathological hallmarks of PD, confirmed its role in PD pathogenesis. Pathological aggregation of the protein might be responsible for neurodegeneration and soluble oligomers of alpha-synuclein are hypothesised to be even more toxic (Lucking CB and Brice, A, Alpha-synuclein and Parkinson's disease Cell Mol Life Sci. 2000 December;57(13-14):1894-908).

Polyneuropathies

In another, equally preferred embodiment of the present invention, a method for treatment of polyneuropathies is provided. Polyneuropathies are defined herein as diseases of the nerves, which often take the form of a noninflammatory degenerative disease of nerves, usually caused by toxins. As an example of these toxic substance, in acute motor axonal neuropathy (AMAN) (Kornberg, A. J. and Pestronk, A., Muscle Nerve 17:100-104 (1994)) and Miller-Fisher syndrome (Chiba, A. et al., Ann. Neurol. 31:677-679 (1992)), antibodies directed against neural antigens, such as glycolipids, have been reported in 30% to 90% of patients. Methods disclosed of the present invention are envisaged as being capable of treating any polyneuropathy caused, or associated with, toxic substances.

Multiple Sclerosis

In another, equally preferred embodiment of the present invention, a method for treatment of Multiple Sclerosis is provided. The “pathogen-mediated” theory of multiple sclerosis postulates that pathogens are involved in the etiology of the disease, which has been supported by results showing an association between C. Pneumoniae in the CSF and Multiple Sclerosis (BioDrugs 2001;15(3):199-206). Other diseases may also be linked to toxic substances, including myasthenia gravis, muscular dystrophy, polymyositis, dermatomyositis, dystrophy myotonic and other myotonic syndromes, Amyotrophic lateral sclerosis (ALS), brain tumors, Guillain-Barre-Syndrome, and the like.

In the most preferred embodiment of the present invention, the individual to be treated suffers from Alzheimer's disease.

Working against the prejudice in the art requiring pressure regulatory systems to aid the flow of toxic proteins in prior art shunts, the inventors have made the surprising discovery that this type of active pressure control mechanism is not required for control of CSF flow if the sagittal sinus or transverse sinus is used as the recipient site for CSF comprising toxic substances.

Instead, the flow of CSF can be controlled by the maintenance of a constant resistance to flow within the shunt. Surprisingly, there is only little or no adhesion of toxic proteins to the shunt components using the methods disclosed herein.

Adhesion of toxic proteins is reduced by i) increasing the diameter of the shunt's internal flow-restricting passage—as compared to VP shunts used for shunting CSF comprising toxic substances—while at the same time ii) decreasing the distance that the toxic CSF substances must be transported between the ventricles and the resorption site.

Also, posture-related pressure changes across the shunt are beneficially avoided using the present shunting methods. An optional non-stick coating may be applied to the shunt to further decrease adhesion of toxic proteins.

While CSF is naturally absorbed and removed from circulation, it is presently believed that certain toxic substances which may be present in the CSF, such as those associated with Alzheimer's disease, may accumulate or persist to an extent which can cause Alzheimer's disease or other disorders.

Such substances are either produced in excess and/or are removed at a rate slower than their production rate so that they accumulate and increase in toxicity and/or reach a threshold concentration in which they become toxic within the CSF space.

One aspect of the present invention is directed at methods for the improved removal of such toxic substances from the CSF in order to treat, inhibit, or ameliorate conditions associated with such toxic materials.

In particular, the present invention is directed at reducing the concentration of such substances in CSF by removing portions of the CSF from the CSF space. Such removal is believed to either enhance production of the CSF and/or reduce the natural absorption of the CSF so that the total volume of CSF in the CSF space is not reduced below a safe level. Moreover, the rates at which the CSF is removed are generally quite low (when compared to the rates of removal for treatment of the hydrocephalus) so that the likelihood of removing excessive amounts of CSF is very low.

By removing CSF from the CSF space, the toxic substances present in the removed CSF will thus be removed from the CSF space and will not be available for absorption or re-circulation. As long as the rate of removal exceeds the rate of production of such substances, the concentration of such substances can be reduced. The removed CSF is directed to a natural disposal site in the sagittal sinus, whereby the toxic substance can be better tolerated within the patient's body.

From measurements in 333 patients (Børgesen and Gjerris 1987) and 52 normal humans (Albeck, Børgesen et al.) it has been possible to establish the relationship between CSF production rate (FR), intracranial pressure (ICP), pressure in the sagittal sinus (Pss) and the resistance to outflow of CSF (Rout):
ICP=FR×Rout+PSS

The relation between the intracranial pressure and the formation rate is linear, and the production rate measured was found to be 0.3 ml/min. (Børgesen and Gjerris 1989). The detailed knowledge on CSF-dynamics, obtained in the laboratories at the Department of Neurosurgery, Rigshospitalet, Copenhagen, Denmark, has provided the necessary data which make it possible to define a CSF shunt system that imitates the normal, physiological drainage of CSF.

The present invention thus provides a method for shunting that diverts the CSF comprising toxic substances into its normal resorption site, and the pressure difference over the CSF shunt system used is preferably similar to a relatively low physiological pressure differences between the ventricles and the resorption site, thus regulating the CSF flow to be within the low to normal range and avoiding complications due to hyperdrainage.

An important feature of the method according to the present invention is the maintainence of an essentially constant resistance to flow within the shunt, said constant resistance to flow being independent of the orientation of said shunt main body means. This means that the resistance is independent of whether the person using the shunt system is standing up or lying down.

By using a shunt which exerts a substantially constant resistance to outflow at the low to normal level, and by using the sagittal and/or transverse sinus as the resorption site, the drainage of CSF comprising toxic substances is regulated by the physiological pressure differences between the production site and the resorption site(s).

Excessive increases of the intracranial pressure are paralleled by increases also in the sinus system being used as the resorption site, and the CSF outflow through the shunt is impeded by a resistance in the low to normal range. Overdrainage, which is the most frequent reason for shunt failure in conventional VP shunts, is thus also avoided.

By using the sagittal sinus or transverse sinus as the recipient site, physiological increases of the intracranial pressure will not increase the differential pressure over the shunt. Posture related changes in the differential pressure as seen in shunts leading the CSF to the right atrium of the heart or to the peritoneal cavity are completely avoided.

### DESCRIPTION OF DRAWINGS

FIG. 1 is a longitudinal sectional view of an embodiment of the shunt system used according to the invention,

FIG. 2 is a sectional view of the shunt body shown in FIG. 1,

FIG. 3 is an end view of the shunt body shown in FIG. 2,

FIG. 4 is a longitudinal sectional view of the shunt body taken at right angles to the section shown in FIG. 2,

FIG. 5 is a perspective view of the shunt body shown in FIGS. 2-4,

FIG. 6 is a partial cross-sectional view of the head of a person, in which the shunt system illustrated in FIGS. 1-5 has been installed,

FIG. 7 is a longitudinal sectional view of the head of a person, in which the shunt system illustrated in FIGS. 1-5 has been installed, and

FIG. 8 is a sectional view as that shown in FIG. 7, where the sinus catheter has been inserted in the transverse sinus.

FIG. 9 is a longitudinal sectional view of the head of a person, in which the shunt system illustrated in FIGS. 1-5 has been installed.

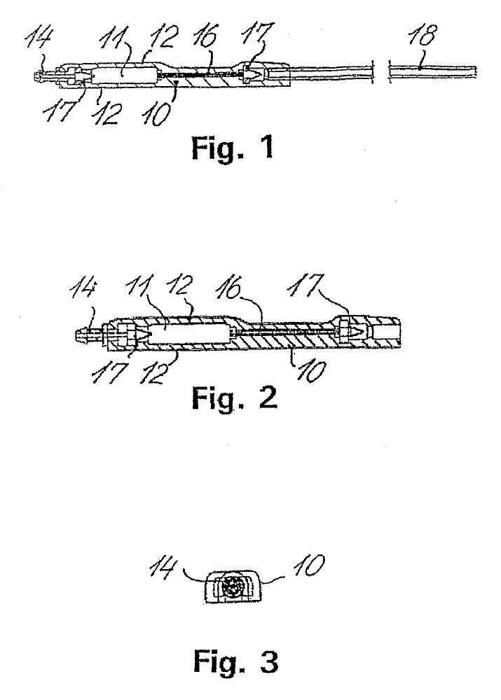

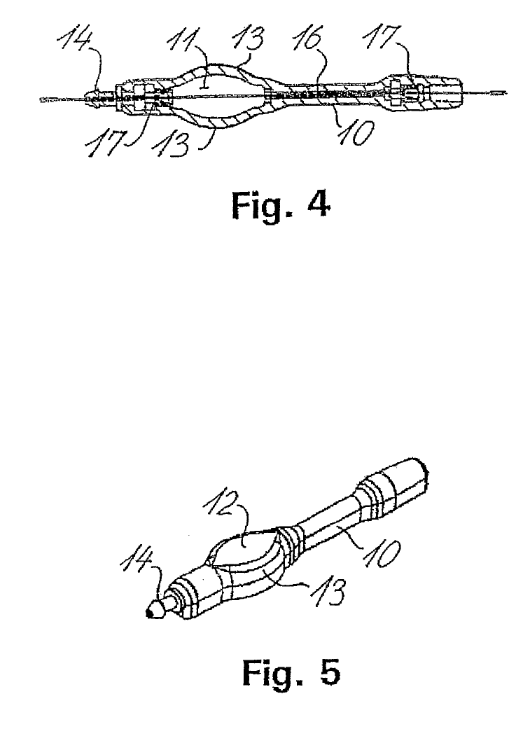

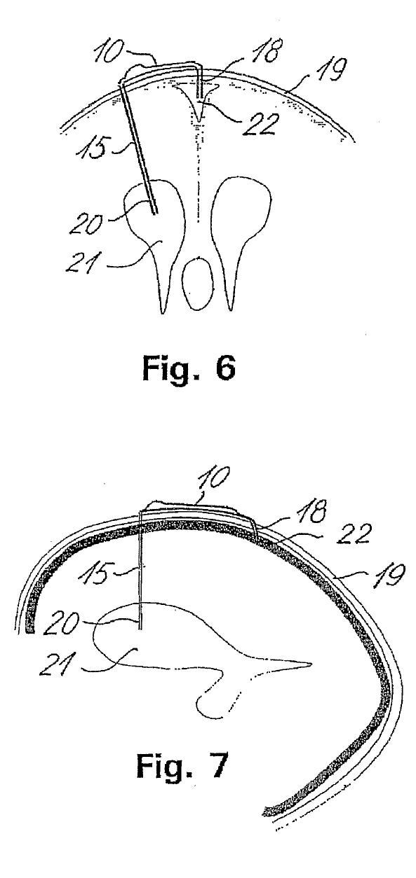

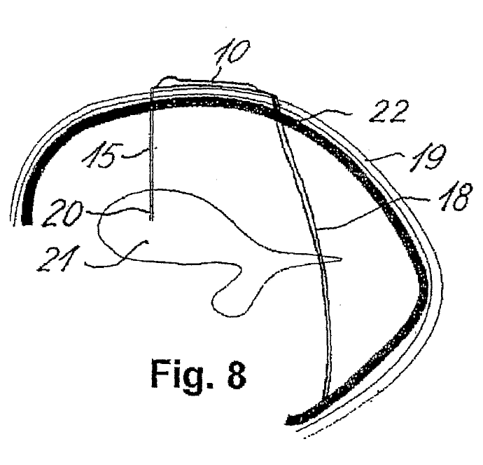

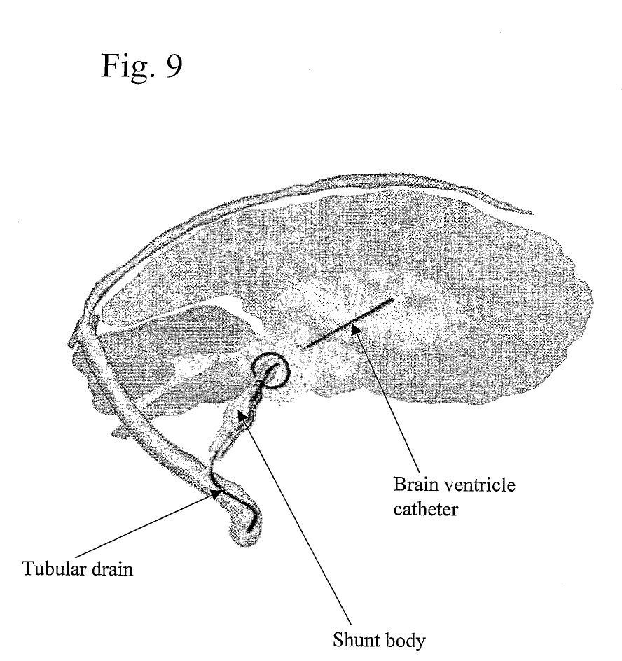

### DETAILED DESCRIPTION OF THE INVENTION

In one embodiment of the present invention, a method is provided for shunting toxic substances present in a brain ventricle to the sinus system of an individual suffering from, or at risk of developing, a condition related to the retention and/or accumulation of toxic substances in the CSF.

It is envisaged that this invention can be used for treating any condition resulting from the accumulation of toxic substances in the patient's brain, for example Alzheimer's disease, Down's Syndrome, hereditary cerebral hemorrhage with amyloidosis of the Dutch-Type (HCHWA-D), epilepsy, Parkinson's disease, polyneuropathies, multiple sclerosis, amyotrophic lateral sclerosis (ALS), myasthenia gravis, muscular dystrophy, dystrophy myotonic, other myotonic syndromes, polymyositis, dermatomyositis, brain tumors, Guillain-Barre-Syndrome, and the like.

Most preferably, the condition treated or prevented by the method of the present invention is Alzheimer's disease.

It is envisaged that the methods herein are useful for treating or preventing a disease caused by any genetic or environmental factor that leads to the generation of an increase in levels of a toxic substance in the CSF. Preferably, said toxic substance is A-beta-42. Equally preferably, said toxic substance is tau. Equally preferably, said toxic substance is beta-2 microglobulin. Equally preferably, said toxic substance is APP. Equally preferably, said toxic substance is a mutant form of APP. Equally preferably, said toxic substance is a neurotrophic factor. Equally preferably, said toxic substance is glutamate or another neurotransmitter. Equally preferably, said toxic substance comprises calcium ions or other bioactive ions. Equally preferably, said toxic substance is alpha-synuclein. Equally preferably, said toxic substance is mutant alpha synuclein. Equally preferably, said toxic substance comprises soluble oligomers of alpha-synuclein. Equally preferably, said toxic substance is an antibody. Equally preferably, said toxic substance is an autoimmune antibody. Equally preferably, said toxic substance is an antibody capable of binding a neural antigen, such as a glycolipid. Equally preferably, said toxic substance is a toxin from a pathogen, such as a virus, fungus, parasite or bacteria, such a C. Pneumoniae. Equally preferably, said toxic substance is an antibody capable of binding a toxin from a pathogen, such as a virus, fungus, parasite or bacteria, such a C. Pneumoniae. In one embodiment of the present invention, the methods shunt more than one toxic substance.

Working against the prejudice in the art requiring pressure regulatory systems to aid the flow of toxic proteins in prior art shunts, the inventors have made the surprising discovery that this type of active pressure control mechanism is not required for control of CSF flow if the sagittal sinus or transverse sinus is used as the recipient site. Instead, the flow of CSF can be controlled by the maintenance of a constant resistance to flow within the shunt. Surprisingly, this does not lead to disadvantageously high adhesion levels of toxic proteins to the shunt using the methods disclosed herein. Adhesion of toxic proteins is reduced by increasing the diameter of the shunt's internal flow-restricting passage and also decreasing the distance that the CSF must be transported between the ventricles and the donor site. Posture-related pressure changes across the shunt are also beneficially avoided. An optional non-stick coating may be applied to the shunt to further decrease adhesion of toxic proteins.

The toxic substance may be in any form capable of being effectively shunted, however preferably said toxic substance is present either in a plaque or in soluble form.

In one embodiment of the present invention, the individual treated using the present invention is male. In an equally preferred embodiment, said individual is female. In another, equally preferred embodiment, the individual treated has a history of head trauma.

In a preferred embodiment of the present invention, the condition treated has an age-related onset, so the individuals treated are at least 50 years old, such as older than 55 years old, such as at least 60 years old, such as older than 65, for example older than 70, such as older than 75, for example older than 80, such as older than 85, for example older than 90, such as older than 95, for example older than 100 years old. Preferably said condition with an age-related onset is Alzheimer's disease.

However, in another, equally preferred, embodiment, said condition may be present in an individual of from 0-120 years old, for instance older than 10 years old, for instance older than 20 years old, for example older than 30 years old, such as older than 35 years old, for example older than 40 years old, for example older than 45 years old, such as older than 50 years old, for example older than 55 years old, such as older than 60 years old, for example older than 65 years old, such as older than 70 years old, for example older than 75 years old.

Preferably, said condition is linked to a genetic aberration, Preferably, said genetic aberration is a mutation in the gene for presenilin 1. Equally preferred is a mutation in the gene APP. Equally preferred is a mutation in the gene for presenilin 2. Equally preferred is a mutation in the gene for apolipoprotein E epsilon4. Equally preferred is trisomy 21, or another genetic aberration linked to chromosome 21 that causes Down's syndrome. It is preferred that the disease linked to a genetic aberration is Alzheimer's disease, and may be early-onset Alzheimer's disease.

The methods disclosed herein are also envisaged as being used in combination with other medical treatments, for instance conventional drug treatments. By “in combination”, it is meant that the methods disclosed herein may be used on an individual prior to, during, or after treatment of the individual with one or more other medical treatment. For example, in one preferred embodiment, an individual is treated with the methods disclosed herein, in combination with administration of one or more of an antidepressant (such as selective serotonin re-uptake inhibitors or trazodone), memantine, ginkgo biloba, selegilene, lazabemide or another drug affecting the monoamine oxidase system, antioxidants, xanthine derivatives (such as Neotrofin), vitamin E, oestrogens, non-steroidal anti-inflammatory drugs, muscarinic agonists, nicotinic agonists, amyloid metabolism modifiers, rivastigmine, aspirin, beta-amyloid “vaccines”, and cholinesterase inhibitors, such as tacrine, donepezil, rivastigmine or galantamine.

In another preferred embodiment, said medical treatment involves a surgical procedure, such as removal of part of the brain of the individual being treated. Said removal is preferably of a brain tumour.

In the methods disclosed herein for shunting toxic substances, the first step is to provide a shunt system for shunting cerebrospinal fluids comprising toxic substances from a brain ventricle to the sinus system of an individual.

There is also provided the use of a shunt body comprising a flow restricting component capable of maintaining a passive and essentially constant resistance to outflow of CSF through the shunt body, in the manufacture of a shunt system for shunting toxic substances present in brain tissue and/or the CSF space to the sinus system of an individual suffering from, or at risk of developing, a condition related to the retention and/or accumulation of toxic substances in brain tissue and/or the CSF space.

Shunt System

The shunt system provided in the present invention comprises a shunt body allowing fluid communication between a brain ventricle and a part of the sinus system of the individual. Said shunt body comprises a flow restricting component capable of maintaining a passive and essentially constant resistance to flow of cerebrospinal fluids through the shunt body. Preferably, said essentially constant resistance to flow of cerebrospinal fluids through the flow restricting component is of a constant value of less than 8 mm Hg/ml/min.

Said shunt system also comprises a brain ventricle catheter capable of being connected to the shunt body at a first location thereof. The brain ventricle catheter is capable of draining cerebrospinal fluids from a brain ventricle to the shunt body. Said shunt system also comprises a sinus catheter capable of being connected to the shunt body at a second location thereof. Said sinus catheter is capable of draining to the sinus system of the individual cerebrospinal fluids having been drained from a brain ventricle and passed through the flow restricting component of the shunt body to the sinus catheter. In one aspect of the present invention, any biocompatible materials capable of allowing CSF flow through the catheters are suitable for use in the brain ventricle catheter and/or sinus catheter; more preferably said brain ventricle catheter and/or sinus catheter is comprised of an adhesion-resistant and/or infection-resistant material. Example of preferred materials include one or more of: a silicone elastomer, polypropylene, polysulfone, nylon or polyethersulfone.

In another preferred embodiment, either all or part of i) the internal or external surface of the shunt body, or either all or part of ii) the internal or external surface of the brain ventricle catheter, or either all or part of iii) the internal or external surface of the sinus catheter, can comprise a biocompatible and/or hemocompatible material comprising an inert surface preventing biological material from maintaining contact with the inert surface, and/or comprising a hemocompatible surface coated with a plurality of charged species capable of increasing the hemocompatibility of the surface.

To carry out the method provided in the present invention, the brain ventricle catheter of the shunt is inserted a brain ventricle of an individual. Furthermore, the sinus catheter of the shunt system is inserted into the sinus system of said individual. The brain ventricle catheter is connected to the shunt body at a first location thereof, and the sinus catheter is connected to the shunt body at a second location thereof. The final step in the method of the present invention comprises shunting toxic substances present in a brain ventricle to the sinus system of the individual.

Flow Restricting Component

In one embodiment of the present invention, the flow restricting component is any structure capable of maintaining a passive and essentially constant resistance to CSF flow. Preferably, the flow restricting component of the shunt body is capable of maintaining a passive and essentially constant resistance to flow of cerebrospinal fluids through the shunt body of from 0.1 to preferably less than 8 mm Hg/ml/min. In another preferred embodiment, the flow restricting component of the shunt body is capable of maintaining a passive and essentially constant resistance to flow of cerebrospinal fluids through the shunt body of from 0.5 to less than 8 mm Hg/ml/min. In another, equally preferred embodiment, the flow restricting component of the shunt body is capable of maintaining a passive and essentially constant resistance to flow of cerebrospinal fluids through the shunt body of from 1 to less than 8 mm Hg/ml/min. In another, equally preferred embodiment, the flow restricting component of the shunt body is capable of maintaining a passive and essentially constant resistance to flow of cerebrospinal fluids through the shunt body of from 2 to less than 8 mm Hg/ml/min. In another, equally preferred embodiment, the flow restricting component of the shunt body is capable of maintaining a passive and essentially constant resistance to flow of cerebrospinal fluids through the shunt body of from 3 to less than 8 mm Hg/ml/min. In another, equally preferred embodiment, the flow restricting component of the shunt body is capable of maintaining a passive and essentially constant resistance to flow of cerebrospinal fluids through the shunt body of from 4 to less than 8 mm Hg/ml/min. In another, equally preferred embodiment, the flow restricting component of the shunt body is capable of maintaining a passive and essentially constant resistance to flow of cerebrospinal fluids through the shunt body of from 6 to less than 8 mm Hg/ml/min. In another, equally preferred embodiment, the flow restricting component of the shunt body is capable of maintaining a passive and essentially constant resistance to flow of cerebrospinal fluids through the shunt body of from 0.1 to 7 mm Hg/ml/min. In another, equally preferred embodiment, the flow restricting component of the shunt body is capable of maintaining a passive and essentially constant resistance to flow of cerebrospinal fluids through the shunt body of from 0.1 to 6 mm Hg/ml/min. In another, equally preferred embodiment, the flow restricting component of the shunt body is capable of maintaining a passive and essentially constant resistance to flow of cerebrospinal fluids through the shunt body of from 0.1 to 5 mm Hg/ml/min. In another, equally preferred embodiment, the flow restricting component of the shunt body is capable of maintaining a passive and essentially constant resistance to flow of cerebrospinal fluids through the shunt body of from 0.1 to 4 mm Hg/ml/min. In another, equally preferred embodiment, the flow restricting component of the shunt body is capable of maintaining a passive and essentially constant resistance to flow of cerebrospinal fluids through the shunt body of from 0.1 to 3 mm Hg/ml/min. In another, equally preferred embodiment, the flow restricting component of the shunt body is capable of maintaining a passive and essentially constant resistance to flow of cerebrospinal fluids through the shunt body of from 0.1 to 2 mm Hg/ml/min. In another, equally preferred embodiment, the flow restricting component of the shunt body is capable of maintaining a passive and essentially constant resistance to flow of cerebrospinal fluids through the shunt body of from 0.1 to 1 mm Hg/ml/min. In another, equally preferred embodiment, the flow restricting component of the shunt body if capable of maintaining a passive and essentially constant resistance to flow of cerebrospinal fluids through the shunt body of from such as from 1 to 7 mm Hg/ml/min. In another, equally preferred embodiment, the flow restricting component of the shunt body is capable of maintaining a passive and essentially constant resistance to flow of cerebrospinal fluids through the shunt body of from 1 to 5 mm Hg/ml/min. In another, equally preferred embodiment, the flow restricting component of the shunt body is capable of maintaining a passive and essentially constant resistance to flow of cerebrospinal fluids through the shunt body of from 1 to 3 mm Hg/ml/min. In another, equally preferred embodiment, the flow restricting component of the shunt body is capable of maintaining a passive and essentially constant resistance to flow of cerebrospinal fluids through the shunt body of from 1 to 2 mm Hg/ml/min. In another, equally preferred embodiment, the flow restricting component of the shunt body is capable of maintaining a passive and essentially constant resistance to flow of cerebrospinal fluids through the shunt body of from 2 to 7 mm Hg/ml/min. In another, equally preferred embodiment, the flow restricting component of the shunt body is capable of maintaining a passive and essentially constant resistance to flow of cerebrospinal fluids through the shunt body of from 2 to 6 mm Hg/ml/min. In another, equally preferred embodiment, the flow restricting component of the shunt body is capable of maintaining a passive and essentially constant resistance to flow of cerebrospinal fluids through the shunt body of from 2 to 5 mm Hg/ml/min. In another, equally preferred embodiment, the flow restricting component of the shunt body is capable of maintaining a passive and essentially constant resistance to flow of cerebrospinal fluids through the shunt body of from 1 to 4 mm Hg/ml/min. In another, equally preferred embodiment, the flow restricting component of the shunt body is capable of maintaining a passive and essentially constant resistance to flow of cerebrospinal fluids through the shunt body of from 4 to less than 8 mm Hg/ml/min.

In another, equally preferred embodiment, the flow restricting component of the shunt body is capable of maintaining a passive and essentially constant resistance to flow of cerebrospinal fluids through the shunt body of a constant value of 0.1 to 0.5 mm Hg/ml/min, such as from 0.5 to 1.0 mm Hg/ml/min, for example from 1.0 to 1.5 mm Hg/ml/min, such as from 1.5 to 2.0 mm Hg/ml/min, for example from 2.0 to 2.5 mm Hg/ml/min, such as from 2.5 to 3.0 mm Hg/ml/min, for example from 3.0 to 3.5 mm Hg/ml/min, such as from 3.5 to 4.0 mm Hg/ml/min, for example from 4.0 to 4.5 mm Hg/ml/min, such as from 4.5 to 5.0 mm Hg/ml/min, for example from 5.0 to 5.5 mm Hg/ml/min, such as from 5.5 to 6.0 mm Hg/ml/min, for example from 6.0 to 6.5 mm Hg/ml/min, such as from 6.5 to 7.0 mm Hg/ml/min, for example from 7.0 to 7.5 mm Hg/ml/min, such as from 7.5 to less than 8.0 mm Hg/ml/min, for example from 0.1 to 1 mm Hg/ml/min, such as from 1 to 2 mm Hg/ml/min, for example from 2 to 3 mm Hg/ml/min, such as from 3 to 4 mm Hg/ml/min, for example from 4 to 5 mm Hg/ml/min, such as from 5 to 6 mm Hg/ml/min, for example from 6 to 7 mm Hg/ml/min, such as from 7 to less than 8 mm Hg/ml/min, for example from 0.1 to 2 mm Hg/ml/min, such as from 2 to 4 mm Hg/ml/min, for example from 4 to 6 mm Hg/ml/min, such as from 6 to less than 8 mm Hg/ml/min, for example from 0.1 to 2.5 mm Hg/ml/min, such as from 2.5 to 5.0 mm Hg/ml/min, for example from 5.0 to 7.5 mm Hg/ml/min, such as from 3.0 to 7.0 mm Hg/ml/min, for example from 3.5 to 6.5 mm Hg/ml/min, such as from 4.0 to 6.0 mm Hg/ml/min, for example from 4.5 to 5.5 mm Hg/ml/min, such as about 5.0 mm Hg/ml/min.

Preferably, the flow restricting component of the shunt body is selected from the group consisting of a tubular structure, a plurality of tubular structures, a porous mass, a fibrous mass, a structure being restricted by co-extending fibres arranged therein, and a structure being restricted by co-extending rods arranged therein, although any structure capable of maintaining a constant resistance to flow is envisaged as being within the scope of the present invention. In one embodiment, said flow restricting component may be made from one or more material capable of maintaining a passive and essentially constant resistance to flow; more preferably said brain ventricle catheter and/or sinus catheter is comprised of an adhesion-resistant and/or infection-resistant material. More preferably, said material is biocompatible. Example of preferred materials include one or more of: a silicone elastomer, HD polyethylene, such as gas sterilized polypropylene, polysulfone, polystyrene, PVC, nylon, titanium or polyethersulfone. The material may be coated with a material, such as teflon or a turbostratic carbon, such as pyrolytic carbon. Said material is preferably biocompatible and/or non-stick.

The length of the flow restricting compartment is important for generating the desired level of resistance to flow, and can be calculated according to the law of Hagen-Poiseulle taking into consideration the required resistance to CSF-outflow. In particularly preferred embodiments, the internal radius of the tubular flow passage restricting means is more than 0.05 mm and preferably less than 0.50 mm, for example a tubular structure having an internal radius of about 0.06 mm, for example about 0.07 mm, such as about 0.08 mm, for example about 0.09 mm, such as about 0.10 mm, for example about 0.11 mm, such as about 0.12 mm, for example about 0.13 mm, such as about 0.14 mm, for example about 0.15 mm, such as about 0.16 mm, for example about 0.17 mm, such as about 0.18 mm, for example about 0.19 mm, such as about 0.20 mm, for example about 0.21 mm, such as about 0.22 mm, for example about 0.23 mm, such as 0.24 mm, for example 0.25 mm, such as 0.26 mm, for example 0.27 mm, for example about 0.28 mm, such as about 0.29 mm, for example about 0.30 mm, such as 0.31 mm, for example 0.32 mm, such as 0.33 mm, for example 0.34 mm, for example about 0.35 mm, such as about 0.36 mm, for example about 0.37 mm, such as 0.38 mm, for example 0.39 mm, such as 0.40 mm, for example 0.42 mm, for example about 0.44 mm, such as about 0.46 mm, for example a tubular structure having an internal radius of about 0.48 mm. In another embodiment, the flow restricting component of the shunt body comprises a single tubular structure having an internal diameter of less than 0.2 mm.

Appropriate lengths of the flow restricting component can be calculated accordingly, as follows:
L=((ICP−Pss)×7×pi×R⁴)/8×F×V (Hagen-Poiseulle's law), wherein ICP is the intracranial pressure, Pss is the pressure in the sagittal sinus, F is the flow rate of the cerebrospinal fluid and V is the viscosity of the cerebrospinal fluid.

In one preferred embodiment, the length of the flow restricting component is in the range of from about 3.0 mm to about 90 mm, such as from about 3.0 mm to about 80 mm, for example from about 3.0 mm to about 75 mm, such as from about 3.0 mm to about 70 mm, for example from about 3.0 mm to about 65 mm, such as from about 3.0 mm to about 60 mm, for example from about 3.0 mm to about 55 mm, such as from about 3.0 mm to about 50 mm, for example from about 3.0 mm to about 45 mm, such as from about 3.0 mm to about 40 mm, for example from about 3.0 mm to about 35 mm, such as from about 3.0 mm to about 30 mm, for example from about 3.0 mm to about 25 mm, such as from about 3.0 mm to about 22 mm, for example from about 3.0 mm to about 20 mm, such as from about 3.0 mm to about 18 mm, for example from about 3.0 mm to about 16 mm, such as from about 3.0 mm to about 14 mm, for example from about 3.0 mm to about 12 mm, such as from about 3.0 mm to about 10 mm, for example from about 10 mm to about 90 mm, such as from about 10 mm to about 80 mm, for example from about 10 mm to about 75 mm, such as from about 10 mm to about 70 mm, for example from about 10 mm to about 65 mm, such as from about 10 mm to about 60 mm, for example from about 10 mm to about 55 mm, such as from about 10 mm to about 50 mm, for example from about 10 mm to about 45 mm, such as from about 10 mm to about 40 mm, for example from about 10 mm to about 35 mm, such as from about 10 mm to about 30 mm, for example from about 10 mm to about 25 mm, such as from about 10 mm to about 20 mm, for example from about 10 mm to about 15 mm, such as about 10 mm, for example about 15 mm, such as about 20 mm, for example about 22 mm, such as about 24 mm, for example about 26 mm, such as about 20 mm, for example about 22 mm, such as about 24 mm, for example about 26 mm, such as about 28 mm, for example about 30 mm, such as about 32 mm, for example about 34 mm, such as about 36 mm, for example about 38 mm, such as about 40 mm, for example about 45 mm, such as about 50 mm, for example about 55 mm, such as about 60 mm, for example about 65 mm, such as about 70 mm, for example about 75 mm, such as about 80 mm, for example about 85 mm.

In another embodiment of the present invention, the total length of the at least one tubular structure of the flow restricting component is divided into two or more individual segments.

Shunt Location

In one embodiment of the present invention, cerebrospinal fluid is shunted from a brain ventricle to either or both of the two large venous sinuses of the cranium that begin at the bony protuberance on the middle of the inner surface of the occipital bone at the intersection of its bony ridges and terminate at the jugular foramen on either side. More preferably, the cerebrospinal fluid is shunted from a brain ventricle to the sagittal sinus. In an equally preferred embodiment of the present invention, the cerebrospinal fluid is shunted from the brain ventricle and to the transverse sinus.

Shunt Body

In one preferred embodiment of the present invention, the shunt body of the shunt system comprises at least one check valve for preventing cerebrospinal fluid present in the sinus catheter or cerebrospinal fluid, having been shunted to the sinus system of the individual, from flowing back from the sinus catheter or from the sinus system to the shunt body or to the brain ventricle catheter. Preferably, said at least one check valve does not have any inherent resistance or opening pressure, and essentially does not exert any resistance on the flow of cerebrospinal fluid from the brain ventricle catheter through the shunt body to the sinus catheter.

More preferably, the resistance to flow through the shunt body is independent of the at least one check valve and defined solely by the flow resistance of the flow restricting component. In the most preferred embodiment, the operation of the at least one check valve is independent of a predetermined opening pressure to be overcome by the differential pressure defined by the difference between the intracranial pressure and the pressure in the sinus.

Preferably, the at least one check valve comprises a ball valve and optionally further comprises valve members selected from the group consisting of guided rigid valve members and flexible valve members, including rigid, ring shaped valve members, and flexible valve members such as tongue-shaped laminae. In one preferred embodiment of the present invention, the at least one check valve comprises a mitral silicone valve.

Preferably, said check valve comprises components made out of one or more of rubber, Stellite alloy, titanium, stainless steel, turbostatic carbons such as pyrolytic carbon, or silicone rubber components, optionally coated with a biocompatible coating such as titanium nitride or turbostatic carbons such as pyrolytic carbon.

Shunt System

In one preferred embodiment of the present invention, the method comprises the further steps of connecting the brain ventricle catheter to a first end location of the shunt body, and connecting the sinus catheter to a second end location of said shunt body. The shunt system in one embodiment preferably comprises a shunt body (10), preferably made from silicone rubber, an antechamber (11) having opposite flat walls (12), preferably made from hard silicone rubber, and opposite domed walls (13), preferably made from soft, perforatable, self-healing silicone rubber.

Preferably, at the proximal end (the top end) of the shunt body, the chamber walls end in a tapering end comprising a tip (14), to which a brain ventricle catheter (15) can be connected and secured. Preferably, the antechamber (11) is connected to the tubular flow restricting component (16) so that the distal end of the chamber (11) forms an inlet to a tubular flow restricting component (16).

Preferably, at least one check valve or non-return valve (17) is arranged both at the entrance to the antechamber (11) and at the outlet of the tubular flow restricting component (16). In one preferred embodiment of the present invention, fluidic connection to the sinus of the individual is provided by a tubular drain (18), and fluidic connection to a brain ventricle of the individual is provided by a brain ventricle catheter (15). The brain ventricle catheter (15) is preferably attached to the tip or inlet connector (14), which is provided with an annular bead, and the brain ventricle catheter is optionally secured by means of a ligature.

Preferably, the length of the connector (14) is about 5 mm. In one preferred embodiment of the present invention, the tubular flow restricting component (16) is dimensioned in accordance with Hagen-Poiseulle's law so as to provide a passive and substantially constant resistance to flow of less than 8 mm Hg/ml/min. Preferably, the tubular flow restricting component is substantially straight. Preferably, the inner walls of the flow restricting component are substantially smooth. The material from which the walls of the tubular flow restricting component are made is preferably selected from the group consisting of hard silicone rubber, HD polyethylene, such as gas sterilized polypropylene, polycarbonate, polysulfone, polystyrene, PVC and titanium. The tubular drain (18) for the sinus is preferably made from titanium or silicone rubber.

In one preferred embodiment of the invention, the distal 5 mm of the tubular drain (18) has an outer diameter of 2 mm and an inner diameter of 1.5 mm, and the part of the drain that goes through the skull has generally an outer diameter of 3 mm and an inner diameter of 1.5 mm. Furthermore, it is preferred that the distance of the part of the drain with the largest diameter can be regulated so as to fit the distance from the shunt body to the hole over the sagittal sinus. Preferably the tubular drain (18) comprises a first tube, preferably comprised of titanium tube, an inner diameter of 1.5 mm and a length of about 20 mm, attached to a second tube, preferably comprised of silicone rubber, with and outer/inner diameter of 3/1.5 mm, and a length of about 60 mm.

Preferably, the method of the present invention comprises the further step of guiding the first tube into the sinus through a borehole in the skull of the individual, wherein guidance is achieved by operating a stilet contained in the tubular drain (18).

In a preferred embodiment of the present invention, the flow rate of shunted cerebrospinal fluid is constant. Preferably, said constant flow rate is in the range of from 40 ml per hour to 140 ml per hour. In another preferred embodiment of the present invention, the constant flow rate is about 40 ml per hour, such as about 45 ml/hour, for example 50 ml per hour, such as about 55 ml/hour, for example about 60 ml per hour, such as about 65 ml/hour, for example about 70 ml per hour, such as about 75 ml/hour, for example about 80 ml per hour, such as about 85 ml/hour, for example about 90 ml per hour, such as about 95 ml/hour, for example 100 ml per hour, such as about 105 ml/hour, for example about 110 ml per hour, such as about 115 ml/hour, for example about 120 ml per hour, such as about 125 ml/hour, for example about 130 ml per hour, such as about 135 ml/hour, for example about 140 ml per hour, such as from 40 to 50 ml per hour, for example from 50 to 60 ml per hour, such as from 60 to 70 ml per hour, for example from 70 to 80 ml per hour, such as from 80 to 90 ml per hour, for example from 90 to 100 ml per hour, such as from 110 to 120 ml per hour, for example from 120 to 130 ml per hour, such as from 130 to 140 ml per hour.

Preferably, the intercranial pressure of the individual is in the range of from −170 mm Hg to 200 mm Hg.

Biocompatible Materials

It will be understood that a “biocompatible” material as defined herein is a material which, when inserted into the brain of an individual, is capable of being reasonably well tolerated by the individual's body, i.e said material does not trigger major immune reactions or acute phase responses.

Biocompatibility shall refer equally to materials characterised by an inert surface, such as diamond-like-carbon, preventing biological material from maintaining a longer lasting contact with the inert surface, as well as to a surface, such as a polymer, coated with a plurality of charged species, such as e.g. hydrophilic polyethylene glycols, capable of increasing in particular the hemocompatibility of the polymer. Longer lasting contact as used herein is a contact which results in undesirable attachment to the surface, normally longer lasting contact will be a contact lasting at least hours, such as at least weeks, for example months.

Preferred examples of biocompatible materials are disclosed herein below. Carbon comprising inert materials represent one preferred class of biocompatible materials.

Carbon forms a strongly bonded 3 dimensional network when deposited as a coating under energetic conditions. This amorphous coating has properties approaching those of diamond as regards hardness, friction, chemical inertness and atomic density hence the term diamond like carbon (DLC). DLC coatings can be produced by plasma assisted chemical vapour deposition from hydrocarbon precursor gases, the coatings contain carbon and hydrogen (to about 30%) and therefore consist of elements which are main constituents in living organisms. In vitro tests have shown DLC to be biocompatible (L A Thomson, F G Law, N Rushton, J Franks. Biomaterials 12, 37 (1991)) and in vivo tests indicate that the coating also has hemocompatible properties.

Because of its atomic density, the coating acts as an effective diffusion barrier preventing ions from the shunt entering the body and protecting the shunt from attack by the biological environment. Turbostratic carbons, like pyrolytic carbon, are a form of graphite that is stronger and more wear resistant. Turbostatic carbons such as “On-X Carbon” (made by the “Medical Carbon Research Institute”, MCRI) are highly hemocompatible.

Sputtered carbon coatings such as Graphit-iC give exceptional friction and wear results in simple laboratory tests against metal counterfaces, demonstrating a high load bearing capacity and operating well in water-based environments, as well as being biocompatible.

Many ceramics, such as titanium nitride (TiN), are also known to have beneficial biocompatible and non-stick properties. TiN has been shown in some in vitro tests to be even more hemocompatible than pyrolytic carbon.

Phosphatidyl choline di-ester is another highly biocompatible coating.

Teflon and the like are other non-stick biocompatible materials exhibiting non-stick properties.

In a preferred embodiment of the present invention, either all or part of i) the internal or external surface of the shunt body, or either all or part of ii) the internal or external surface of the brain ventricle catheter, or either all or part of iii) the internal or external surface of the sinus catheter, can comprise a biocompatible/hemocompatible material comprising an inert surface preventing biological material from maintaining contact with the inert surface, and/or comprising a hemocompatible surface coated with a plurality of charged species capable of increasing the hemocompatibility of the surface.

The hemocompatible surface coated with a plurality of charged species capable of increasing the hemocompatibility of the surface can be e.g. a silicone elastomer, teflon, HD polyethylene, such as gas sterilized polypropylene, polysulfone, polystyrene, PVC, nylon, titanium, shape memory alloys such as Nitinol or polyethersulfone. The charged species can be e.g. polyethylene glycols or another macromolecule having a molecular weight of less than e.g. 20.000. The hemocompatible surface is in one embodiment a modified polymer surface as disclosed in PCT/DK00/00065 and PCT/DK01/00557.

The internal or external surfaces of the shunt system are preferably sterilisable. It is preferred that one or more of said surfaces act as an effective diffusion barrier preventing ions from the shunt entering the body and protecting the shunt from attack by the biological environment.

In another preferred embodiment of the present invention, one or more of said surfaces are non-adhesive. In another preferred embodiment, one or more of said surfaces are non-toxic. In another preferred embodiment, one or more of said surfaces are non-immunogenic.

In one preferred embodiment of the present invention, said biocompatible/hemocompatible material comprises diamond like carbon (DLC) or the like. Equally preferably, said biocompatible/hemocompatible material can comprise a turbostratic carbon, more preferably pyrolytic carbon.

In another preferred embodiment of the present invention, said biocompatible/hemocompatible material comprises a ceramic. Said ceramic is preferably titanium nitride (TiN), or the like. In another preferred embodiment, said biocompatible/hemocompatible material comprises phosphatidyl choline di-ester. In another preferred embodiment, said biocompatible/hemocompatible material comprises a Sputtered carbon coating, such as Graphit-iC or the like. In another preferred embodiment, said biocompatible/hemocompatible material comprises Teflon, and the like. In another embodiment of the present invention, said biocompatible/hemocompatible material comprises a calcification-resistant biocompatible material.

1. A method for shunting toxic substances, present in a brain ventricle, to the sinus system of an individual suffering from, or at risk of developing, a condition related to the retention and/or accumulation of toxic substances in brain tissue and/or the CSF space, said method comprising the steps of
i) providing a shunt system for shunting cerebrospinal fluids comprising toxic substances, such as amyloid proteins, from a brain ventricle to the sinus system of an individual, wherein said shunt system comprises
a) a shunt body allowing fluid communication between a brain ventricle and a part of the sinus system of the individual,
wherein said shunt body comprises a flow restricting component capable of maintaining a passive and essentially constant resistance to flow of cerebrospinal fluids through the shunt body,
b) a brain ventricle catheter capable of being connected to the shunt body at a first location thereof,
wherein the brain ventricle catheter is capable of draining cerebrospinal fluids from a brain ventricle to the shunt body, and
c) a sinus catheter capable of being connected to the shunt body at a second location thereof,
wherein the sinus catheter is capable of draining to the sinus system of the individual cerebrospinal fluids having been drained from a brain ventricle and passed through the flow restricting component of the shunt body to the sinus catheter,
wherein optionally either all or part of i) the internal or external surface of the shunt body, or either all or part of ii) the internal or external surface of the brain ventricle catheter, or either all or part of iii) the internal or external surface of the sinus catheter, can comprise a biocompatible/hemocompatible material comprising an inert surface preventing biological material from maintaining contact with the inert surface, and/or comprising a hemocompatible surface coated with a plurality of charged species capable of increasing the hemocompatibility of the surface,
ii) inserting into a brain ventricle of the individual the brain ventricle catheter of the shunt system capable of being connected to the shunt body at a first location thereof,
iii) inserting into the sinus system of the individual the sinus catheter of the shunt system capable of being connected to the shunt body at a second location thereof, and
iv) shunting toxic substances, such as amyloid proteins, present in a brain ventricle to the sinus system of the individual suffering from, or at risk of developing, a condition related to the retention and/or accumulation of toxic substances in brain tissue and/or the CSF space.

2. The method of claim 1, wherein the condition related to the retention and/or accumulation of toxic substances in the CSF is Alzheimer's disease.

3. The method of any of the previous claims, wherein the condition related to the retention and/or accumulation of toxic substances in the CSF is Down's Syndrome.

4. The method of any of the previous claims, wherein the condition related to the retention and/or accumulation of toxic substances in the CSF is hereditary cerebral hemorrhage with amyloidosis of the Dutch-Type (HCHWA-D) or the like.

5. The method of any of the previous claims, wherein the condition related to the retention and/or accumulation of toxic substances in the CSF is epilepsy.

6. The method of any of the previous claims, wherein the condition related to the retention and/or accumulation of toxic substances in the CSF is Parkinson's disease.

7. The method of any of the previous claims, wherein the condition related to the retention and/or accumulation of toxic substances in the CSF is a polyneuropathy.

8. The method of any of the previous claims, wherein the condition related to the retention and/or accumulation of toxic substances in the CSF is selected from one or more of multiple sclerosis, amyotrophic lateral sclerosis (ALS), myasthenia gravis, muscular dystrophy, dystrophy myotonic or another myotonic syndrome, polymyositis, dermatomyositis, a brain tumor or Guillain-Barre-Syndrome.

9. The method of any of claims 1-8, wherein the toxic substance is one or more of tau, beta-2microglobulin or A-beta-42

10. The method of any of claims 1-9, wherein the flow restricting component of the shunt body is capable of maintaining a passive and essentially constant resistance to flow of cerebrospinal fluids through the shunt body of a constant value of from 0.1 to less than 8 mm Hg/ml/min.

11. The method of any of claims 1-9, wherein the flow restricting component of the shunt body is capable of maintaining a passive and essentially constant resistance to flow of cerebrospinal fluids through the shunt body of a constant value of from 0.5 to less than 8 mm Hg/ml/min.

12. The method of any of claims 1-9, wherein the flow restricting component of the shunt body is capable of maintaining a passive and essentially constant resistance to flow of cerebrospinal fluids through the shunt body of a constant value of from 1 to less than 8 mm Hg/ml/min.

13. The method of any of claims 1-9, wherein the flow restricting component of the shunt body is capable of maintaining a passive and essentially constant resistance to flow of cerebrospinal fluids through the shunt body of a constant value of from 2 to less than 8 mm Hg/ml/min.

14. The method of any of claims 1-9, wherein the flow restricting component of the shunt body is capable of maintaining a passive and essentially constant resistance to flow of cerebrospinal fluids through the shunt body of a constant value of from 3 to less than 8 mm Hg/ml/min.

15. The method of any of claims 1-9, wherein the flow restricting component of the shunt body is capable of maintaining a passive and essentially constant resistance to flow of cerebrospinal fluids through the shunt body of a constant value of from 4 to less than 8 mm Hg/ml/min.

16. The method of any of claims 1-9, wherein the flow restricting component of the shunt body is capable of maintaining a passive and essentially constant resistance to flow of cerebrospinal fluids through the shunt body of a constant value of from 6 to less than 8 mm Hg/ml/min.

17. The method of any of claims 1-9, wherein the flow restricting component of the shunt body is capable of maintaining a passive and essentially constant resistance to flow of cerebrospinal fluids through the shunt body of a constant value of from 0.1 to 7 mm Hg/ml/min.

18. The method of any of claims 1-9, wherein the flow restricting component of the shunt body is capable of maintaining a passive and essentially constant resistance to flow of cerebrospinal fluids through the shunt body of a constant value of from 0.1 to 6 mm Hg/ml/min.

19. The method of any of claims 1-9, wherein the flow restricting component of the shunt body is capable of maintaining a passive and essentially constant resistance to flow of cerebrospinal fluids through the shunt body of a constant value of from 0.1 to 5 mm Hg/ml/min.

20. The method of any of claims 1-9, wherein the flow restricting component of the shunt body is capable of maintaining a passive and essentially constant resistance to flow of cerebrospinal fluids through the shunt body of a constant value of from 0.1 to 4 mm Hg/ml/min.

21. The method of any of claims 1-9, wherein the flow restricting component of the shunt body is capable of maintaining a passive and essentially constant resistance to flow of cerebrospinal fluids through the shunt body of a constant value of from 0.1 to 3 mm Hg/ml/min.

22. The method of claim 1, wherein the flow restricting component of the shunt body is capable of maintaining a passive and essentially constant resistance to flow of cerebrospinal fluids through the shunt body of a constant value of from 0.1 to 2 mm Hg/ml/min.

23. The method of any of claims 1-9, wherein the flow restricting component of the shunt body is capable of maintaining a passive and essentially constant resistance to flow of cerebrospinal fluids through the shunt body of a constant value of from 0.1 to 1 mm Hg/ml/min.

24. The method of any of claims 1-9, wherein the flow restricting component of the shunt body is capable of maintaining a passive and essentially constant resistance to flow of cerebrospinal fluids through the shunt body of a constant value of from 1 to 7 mm Hg/ml/min.

25. The method of any of claims 1-9, wherein the flow restricting component of the shunt body is capable of maintaining a passive and essentially constant resistance to flow of cerebrospinal fluids through the shunt body of a constant value of from 1 to 5 mm Hg/ml/min.

26. The method of any of claims 1-9, wherein the flow restricting component of the shunt body is capable of maintaining a passive and essentially constant resistance to flow of cerebrospinal fluids through the shunt body of a constant value of from 1 to 3 mm Hg/ml/min.

27. The method of any of claims 1-9, wherein the flow restricting component of the shunt body is capable of maintaining a passive and essentially constant resistance to flow of cerebrospinal fluids through the shunt body of a constant value of from 1 to 2 mm Hg/ml/min.

28. The method of any of claims 1-9, wherein the flow restricting component of the shunt body is capable of maintaining a passive and essentially constant resistance to flow of cerebrospinal fluids through the shunt body of a constant value of from 2 to 7 mm Hg/ml/min.

29. The method of any of claims 1-9, wherein the flow restricting component of the shunt body is capable of maintaining a passive and essentially constant resistance to flow of cerebrospinal fluids through the shunt body of a constant value of from 2 to 6 mm Hg/ml/min.

30. The method of any of claims 1-9, wherein the flow restricting component of the shunt body is capable of maintaining a passive and essentially constant resistance to flow of cerebrospinal fluids through the shunt body of a constant value of from 2 to 5 mm Hg/ml/min.

31. The method of any of claims 1-9, wherein the flow restricting component of the shunt body is capable of maintaining a passive and essentially constant resistance to flow of cerebrospinal fluids through the shunt body of a constant value of from 1 to 4 mm Hg/ml/min.

32. The method of any of claims 1-9, wherein the flow restricting component of the shunt body is capable of maintaining a passive and essentially constant resistance to flow of cerebrospinal fluids through the shunt body of a constant value of from 4 to less than 8 mm Hg/ml/min.

33. The method of any of claims 1-9, wherein the flow restricting component of the shunt body is capable of maintaining a passive and essentially constant resistance to flow of cerebrospinal fluids through the shunt body of a constant value of from 0.1 to 0.5 mm Hg/ml/min, such as from 0.5 to 1.0 mm Hg/ml/min, for example from 1.0 to 1.5 mm Hg/ml/min, such as from 1.5 to 2.0 mm Hg/ml/min, for example from 2.0 to 2.5 mm Hg/ml/min, such as from 2.5 to 3.0 mm Hg/ml/min, for example from 3.0 to 3.5 mm Hg/ml/min, such as from 3.5 to 4.0 mm Hg/ml/min, for example from 4.0 to 4.5 mm Hg/ml/min, such as from 4.5 to 5.0 mm Hg/ml/min, for example from 5.0 to 5.5 mm Hg/ml/min, such as from 5.5 to 6.0 mm Hg/ml/min, for example from 6.0 to 6.5 mm Hg/ml/min, such as from 6.5 to 7.0 mm Hg/ml/min, for example from 7.0 to 7.5 mm Hg/ml/min, such as from 7.5 to less than 8.0 mm Hg/ml/min, for example from 0.1 to 1 mm Hg/ml/min, such as from 1 to 2 mm Hg/ml/min, for example from 2 to 3 mm Hg/ml/min, such as from 3 to 4 mm Hg/ml/min, for example from 4 to 5 mm Hg/ml/min, such as from 5 to 6 mm Hg/ml/min, for example from 6 to 7 mm Hg/ml/min, such as from 7 to less than 8 mm Hg/ml/min, for example from 0.1 to 2 mm Hg/ml/min, such as from 2 to 4 mm Hg/ml/min, for example from 4 to 6 mm Hg/ml/min, such as from 6 to less than 8 mm Hg/ml/min, for example from 0.1 to 2.5 mm Hg/ml/min, such as from 2.5 to 5.0 mm Hg/ml/min, for example from 5.0 to 7.5 mm Hg/ml/min, such as from 3.0 to 7.0 mm Hg/ml/min, for example from 3.5 to 6.5 mm Hg/ml/min, such as from 4.0 to 6.0 mm Hg/ml/min, for example from 4.5 to 5.5 mm Hg/ml/min, such as about 5.0 mm Hg/ml/min.

34. The method of any of claims 1 to 33 wherein the flow restricting component of the shunt body is selected from the group consisting of a tubular structure, a plurality of tubular structures, a porous mass, a fibrous mass, a structure being restricted by co-extending fibres arranged therein, and a structure being restricted by co-extending rods arranged therein.

35. The method of any of claims 1 to 33 wherein the flow restricting component of the shunt body comprises at least one tubular structure having an internal radius of more than 0.05 mm and preferably less than 0.50 mm, for example a tubular structure having an internal radius of about 0.06 mm, for example about 0.07 mm, such as about 0.08 mm, for example about 0.09 mm, such as about 0.10 mm, for example about 0.11 mm, such as about 0.12 mm, for example about 0.13 mm, such as about 0.14 mm, for example about 0.15 mm, such as about 0.16 mm, for example about 0.17 mm, such as about 0.18 mm, for example about 0.19 mm, such as about 0.20 mm, for example about 0.21 mm, such as about 0.22 mm, for example about 0.23 mm, such as 0.24 mm, for example 0.25 mm, such as 0.26 mm, for example 0.27 mm, for example about 0.28 mm, such as about 0.29 mm, for example about 0.30 mm, such as 0.31 mm, for example 0.32 mm, such as 0.33 mm, for example 0.34 mm, for example about 0.35 mm, such as about 0.36 mm, for example about 0.37 mm, such as 0.38 mm, for example 0.39 mm, such as 0.40 mm, for example 0.42 mm, for example about 0.44 mm, such as about 0.46 mm, for example a tubular structure having an internal radius of about 0.48 mm.

36. The method of any of claims 32 to 35, wherein the flow restricting component of the shunt body comprises a single tubular structure having an internal diameter of less than 0.2 mm.

37. The method of any of claims 34 to 36, wherein the length of the at least one tubular structure of the flow restricting component of the shunt body is in the range of from about 3.0 mm to about 90 mm, such as from about 3.0 mm to about 80 mm, for example from about 3.0 mm to about 75 mm, such as from about 3.0 mm to about 70 mm, for example from about 3.0 mm to about 65 mm, such as from about 3.0 mm to about 60 mm, for example from about 3.0 mm to about 55 mm, such as from about 3.0 mm to about 50 mm, for example from about 3.0 mm to about 45 mm, such as from about 3.0 mm to about 40 mm, for example from about 3.0 mm to about 35 mm, such as from about 3.0 mm to about 30 mm, for example from about 3.0 mm to about 25 mm, such as from about 3.0 mm to about 22 mm, for example from about 3.0 mm to about 20 mm, such as from about 3.0 mm to about 18 mm, for example from about 3.0 mm to about 16 mm, such as from about 3.0 mm to about 14 mm, for example from about 3.0 mm to about 12 mm, such as from about 3.0 mm to about 10 mm, for example from about 10 mm to about 90 mm, such as from about 10 mm to about 80 mm, for example from about 10 mm to about 75 mm, such as from about 10 mm to about 70 mm, for example from about 10 mm to about 65 mm, such as from about 10 mm to about 60 mm, for example from about 10 mm to about 55 mm, such as from about 10 mm to about 50 mm, for example from about 10 mm to about 45 mm, such as from about 10 mm to about 40 mm, for example from about 10 mm to about 35 mm, such as from about 10 mm to about 30 mm, for example from about 10 mm to about 25 mm, such as from about 10 mm to about 20 mm, for example from about 10 mm to about 15 mm, such as about 10 mm, for example about 15 mm, such as about 20 mm, for example about 22 mm, such as about 24 mm, for example about 26 mm, such as about 20 mm, for example about 22 mm, such as about 24 mm, for example about 26 mm, such as about 28 mm, for example about 30 mm, such as about 32 mm, for example about 34 mm, such as about 36 mm, for example about 38 mm, such as about 40 mm, for example about 45 mm, such as about 50 mm, for example about 55 mm, such as about 60 mm, for example about 65 mm, such as about 70 mm, for example about 75 mm, such as about 80 mm, for example about 85 mm.

38. The method of claims 36 or 37, wherein the total length of the at least one tubular structure of the flow restricting component of the shunt body is divided in two or more individual segments.

39. The method of any of the preceding claims comprising the further step(s) of connecting the sinus catheter to the shunt body at a second location thereof, and/or connecting the brain ventricle catheter to the shunt body at a first location thereof.

40. The method of any of the previous claims, wherein cerebrospinal fluid is shunted from a brain ventricle to either or both of the two large venous sinuses of the cranium that begin at the bony protuberance on the middle of the inner surface of the occipital bone at the intersection of its bony ridges and terminate at the jugular foramen on either side.

41. The method of claim 40, wherein the cerebrospinal fluid is shunted from the brain ventricle and to the sagittal sinus.

42. The method of claim 40, wherein the cerebrospinal fluid is shunted from the brain ventricle and to the transverse sinus.

43. The method of any of the previous claims, wherein the shunt body of the shunt system further comprises at least one check valve for preventing cerebrospinal fluid present in the sinus catheter or cerebrospinal fluid having been shunted to the sinus system of the individual from flowing back from the sinus catheter or from the sinus system to the shunt body or to the brain ventricle catheter.

44. The method of claim 43, wherein the at least one check valve of the shunt body does not have any inherent resistance or opening pressure, and essentially does not exert any resistance on the flow of cerebrospinal fluid from the brain ventricle catheter through the shunt body to the sinus catheter.

45. The method of any of claims 43 and 44, wherein the resistance to flow thorugh the shunt body is independent of the at least one check valve and defined solely by the flow resistance of the flow restricting component.

46. The method of any of claims 43 to 45, wherein the operation of the at least one check valve is independent of a predetermined opening pressure to be overcome by the differential pressure defined by the difference between the intracranial pressure and the pressure in the sinus.

47. The method of any of claims 43 to 46, wherein the at least one check valve comprises a ball valve and optionally further comprises valve members selected from the group consisting of guided rigid valve members and flexible valve members, including rigid, ring shaped valve members, and flexible valve members such as tongue-shaped laminae.

48. The method of any of any of claims 43 to 47, wherein the at least one check valve comprises a mitral silicone valve.

49. The method of any of the previous claims comprising the further steps of connecting the brain ventricle catheter to a first end location of said shunt body, and connecting the sinus catheter to a second end location of said shunt body.

50. The method of any of the previous claims,
wherein the shunt system comprises
a shunt body (10) made from silicone rubber, an antechamber (11) having opposite flat walls (12) made from hard silicone rubber, and opposite domed walls (13) made from soft, perforatable, self-healing silicone rubber,
wherein at the proximal end (the top end) the chamber walls end in a tapering end comprising a tip (14), to which a brain ventricle catheter (15) can be connected and secured,
wherein the antechamber (11) is connected to the tubular flow restricting component (16) so that the distal end of the chamber (11) forms an inlet to a tubular flow restricting component (16),
wherein at least one check valve or non-return valve (17) is arranged both at the entrance to the antechamber (11) and at the outlet of the tubular flow restricting component (16),
wherein fluidic connection to the sinus of the invividual is provided by a tubular drain (18), and
wherein fluidic connection to a brain ventricle of the invividual is provided by a brain ventricle catheter (15).

51. The method of any of the previous claims, wherein the brain ventricle catheter (15) is attached to the tip or inlet connector (14), which is provided with an annular bead, and wherein the the brain ventricle catheter is optionally secured by means of a ligature.

52. The method of any of the previous claims, wherein the length of the connector (14) is about 5 mm.

53. The method of any of claims 50 to 52, wherein the tubular flow restricting component (16) is dimensioned in accordance with Hagen-Poiseulle's law so as to provide a passive and substantially constant resistance to flow of less than 8 mm Hg/ml/min.

54. The method of any of claims 50 to 53, wherein the tubular flow restricting component is substantially straight, and wherein the inner walls of the flow restricting component is substantially smooth.

55. The method of any of claims 50 to 54, wherein the material from which the walls of the tubular flow restricting component is made is selected from the group consisting of hard silicone rubber, HD polyethylene, such as gas sterilized poly propylene, polycarbonate, polysulfone, polystyrene, PVC and titanium.

56. The method of any of claims 50 to 55, wherein the tubular drain (18) for the sinus is made from titanium or silicone rubber.

57. The method of any of claims 50 to 56, wherein the distal 5 mm of the tubular drain (18) has an outer diameter of 2 mm and an inner diameter of 1.5 mm, and wherein the part of the drain that goes through the skull has generally an outer diameter of 3 mm and an inner diameter of 1.5 mm, and wherein the distance of the part of the drain with the largest diameter can be regulated so as to fit the distance from the shunt body to the hole over the sagittal sinus.

58. The method of any of claims 50 to 57, wherein the tubular drain (18) comprises a titanium tube with an inner diameter of 1.5 mm and a length of about 20 mm attached to a silicone rubber tube with and outer/inner diameter of 3/1.5 mm, and a length of about 60 mm.

59. The method of any of the previous claims comprising the further step of guiding the silicone rubber tube into the sinus through a borehole in the skull of the individual, wherein guidance is achieved by operating a stilet contained in the tubular drain (18).

60. The method of any of the previous claims for shunting cerebrospinal fluid, wherein the flow rate of shunted cerebrospinal fluid is constant.

61. The method of any of the previous claims for shunting cerebrospinal fluid, wherein the constant flow rate is in the range of from 40 ml per hour to 140 ml per hour.

62. The method of claim 61 for shunting cerebrospinal fluid, wherein the constant flow rate is about 40 ml per hour, such as about 45 ml/hour, for example 50 ml per hour, such as about 55 ml/hour, for example about 60 ml per hour, such as about 65 ml/hour, for example about 70 ml per hour, such as about 75 ml/hour, for example about 80 ml per hour, such as about 85 ml/hour, for example about 90 ml per hour, such as about 95 ml/hour, for example 100 ml per hour, such as about 105 ml/hour, for example about 110 ml per hour, such as about 115 ml/hour, for example about 120 ml per hour, such as about 125 ml/hour, for example about 130 ml per hour, such as about 135 ml/hour, for example about 140 ml per hour, such as from 40 to 50 ml per hour, for example from 50 to 60 ml per hour, such as from 60 to 70 ml per hour, for example from 70 to 80 ml per hour, such as from 80 to 90 ml per hour, for example from 90 to 100 ml per hour, such as from 110 to 120 ml per hour, for example from 120 to 130 ml per hour, such as from 130 to 140 ml per hour.

63. The method of any of the previous claims for shunting cerebrospinal fluid, wherein the intercranial pressure of the individual is in the range of from −170 mm Hg to 200 mm Hg.

64. The method of any of the previous claims wherein either all or part of i) the internal or external surface of the shunt body, or either all or part of ii) the internal or external surface of the brain ventricle catheter, or either all or part of iii) the internal or external surface of the sinus catheter, can comprise a biocompatible and/or hemocompatible material comprising an inert surface preventing biological material from maintaining contact with the inert surface, and/or comprising a hemocompatible surface coated with a plurality of charged species capable of increasing the hemocompatibility of the surface.

65. The method of claim 64, wherein said biocompatible and/or hemocompatible material is carbon-based.

66. The method of claim 64 wherein said carbon-based biocompatble and/or hemocompatible material comprises or consists of Diamond-Like Carbon (DLC).

67. The method of claim 64 wherein said carbon-based biocompatible and/or hemocompatible material comprises or consists of a turbostratic carbon.

68. The method of claim 64 wherein wherein said carbon-based biocompatible and/or hemocompatible material comprises or consists of a pyrolytic carbon.

69. The method of claim 64 wherein said biocompatible/hemocompatible material comprises or consists of a Sputtered carbon.

70. The method of claim 64, wherein said biocompatible/hemocompatible material comprises or consists of Graphit-iC

71. The method of claim 64, wherein said biocompatible/hemocompatible material comprises or consists of Teflon.

72. The method of claim 64, wherein said biocompatible/hemocompatible material comprises or consists of a Ceramic

73. The method of claim 72, wherein said biocompatible/hemocompatible material comprises or consists of titanium nitride (TiN)

74. The method of claim 64, wherein said biocompatible/hemocompatible material is Phosphatidyl choline di-ester.

75. Use of a shunt body comprising a flow restricting component capable of maintaining a passive and essentially constant resistance to outflow of CSF through the shunt body, in the manufacture of a shunt system for shunting toxic substances present in brain tissue and/or the CSF space to the sinus system of an individual suffering from, or at risk of developing, a condition related to the retention and/or accumulation of toxic substances in brain tissue and/or the CSF space.

76. The use according to claim 75, wherein said condition is according to any of claims 2-8.

77. The use according to any of claims 75-76, wherein said shunt system is according to any of claims 10-38, 43-48, 50-58, 64-74.
